(19) United States
(12) Patent Application Publication
(10) Pub. No.: US 2008/0267938 A1
(43) Pub. Date: Oct. 30, 2008

(54) CARDIAC PHYTO-NUTRACEUTICAL SYNERGISTIC COMPOSITION

(76) Inventor: JOSE ANGEL OLALDE RANGEL, Clearwater, FL (US)

Correspondence Address: JOSE OLALDE RANGEL, 519 CLEVELAND STREET, SUITE 101, CLEARWATER, FL, 33755, US

(21) Appl. No.: 11/531,564

(22) Filed: Sep. 13, 2006

(51) Int. Cl. A61K 31/122; A61P 9/00

(52) U.S. Cl. 424/94.1

(57) ABSTRACT

A Phytoceutical composition for the prevention and treatment of cardiac affections is provided. A specific combination of extracts of plants and nutraceuticals is provided, based on categorizing plants and nutraceuticals into one of three groups, Energy, Bio-Intelligence, and Organization. Such combinations have synergistic effects, with minimal side effects.

### PRIOR RELATED APPLICATIONS

Not applicable.

### FEDERALLY SPONSORED RESEARCH STATEMENT

Not applicable.

### REFERENCE TO MICROFICHE APPENDIX

Not applicable.

### FIELD OF THE INVENTION

The invention relates to a phytoceutical formulation used to treat cardiac disorders and symptoms. The formulation is a particular combination of plants that have synergistic effect in combination. Principles for selecting beneficial formulations are provided.

### BACKGROUND OF THE INVENTION

The academic study of medicinal plants for the treatment of diverse diseases has been nearly as pervasive as the study of Western medicines. The active principles from many traditional medicines have been extracted from plants, the curative agents identified and their mechanisms of action determined. Plant based medicines are typically well tolerated, with less severe side effects as well as a smaller range of side effects. In contrast, while synthetic drugs can be highly effective, their use is often hampered by severe side effects. Additionally, while synthetic pharmaceuticals are based upon single chemicals, many phytomedicines exert their beneficial effects through the additive or synergistic action of several chemical compounds acting at single or multiple target sites associated with a physiological process. As pointed out by Tyler (1999), this synergistic or additive pharmacological effect can be beneficial by eliminating the problematic side effects associated with the predominance of a single xenobiotic compound in the body.

In this respect, Kaufman et al. (1999) extensively documented how synergistic interactions underlie the effectiveness of a number of phytomedicines. A more recent study on a phytomedicine's synergistic effect—Echinacea—is provided by Dalby-Brown et al, 2005. This theme of multiple chemicals acting in an additive or synergistic manner likely has its origin in the functional role of secondary products in promoting plant survival. For example, in the role of secondary products as defense chemicals, a mixture of chemicals having additive or synergistic effects at multiple target sites would not only ensure effectiveness against a wide range of herbivores or pathogens but would also decrease the chances of these organisms developing resistance or adaptive responses (Kaufman et al., 1999; Wink, 1999). Conclusion: On one hand, synthetics may have the required efficacy for disease treatment; however this can be marred by severe side effects. On the other hand, despite the excellent medicinal qualities of many plants, they are individually insufficient to take chronic degenerative diseases into remission. However, there is mounting evidence which demonstrates that medical plants contain synergistic efficacy and/or side-effect neutralizing combinations (Gilani and Rahman, 2005). Thus, what are needed in the art are better treatment regimes with improved patient tolerance, while providing sufficient efficacy.

### SUMMARY OF THE INVENTION

A number of known beneficial plants and tonics were classified according to their capacity to enhance the three main elements that support overall health: Energy (E), Bio-intelligence (I) and Organization (O). A synergistic effect is expected when all three categories of herbs (E, I, O) are included in a formulation, preferably at least two or three or four plants from each category. Thus, the invention provides a composition created using this method of selecting the disease treating formulation according to its principles. Additional formulations are being prepared and tested.

Another embodiment of the invention provides an effective, natural composition for treating high cholesterol and/or triglyceride levels. It can be used—but not limited—to treat: Cardiac insufficiency, ischemic cardiopathy, obstructive hypertrophic myocardiopathy, congestive heart failure and hypertensive cardiopathy. The composition can be used alone, or can be combined with simultaneous use of one or more pharmaceutical compositions.

### DETAILED DESCRIPTION OF THE INVENTION

“Pharmaceutically acceptable excipients” is used herein according to art accepted meanings, and includes those ingredients needed to formulate a medicine for mammalian use, including the use of gelatin capsules.

“Synergistic” or “synergy” is used herein to mean that the effect is more than its additive property. In preferred embodiments, the synergy is at least 1.5, 2, 5, or 10 fold.

By use of “plants,” what is meant herein is that the plant (or that portion with medicinal activity) is used whole, ground, or as an extract. Also included are purified active ingredients and derivatives thereof. However, it is believed that the best efficacy of plants used herein is achieved with the use of the entire plant or its extracts, rather than with the use of isolated active ingredients.

Further, although plants are named here according to commonly used nomenclature, with improving taxonomy plants are often reclassified. Whenever a plant is referenced, it includes related species with similar active ingredients.

The following examples are illustrative only and should not serve to unduly limit the invention.

### EXAMPLE 1

### Plants/Nutraceuticals Characteristics

### Energy Enhancing Components.—

Panax ginseng (Chinese ginseng, panax, ren shen, jintsam, ninjin, Asiatic ginseng, Japanese ginseng, Oriental ginseng, Korean red ginseng) Its main active components are ginsenosides (protopanaxadiols and protopanaxatriols types) have shown a variety of beneficial effects, including anti-inflammatory, antioxidant, and cardiovascular effects.

They also confer energizing properties because they increase ATP synthesis. Ginsenoside-Rg1 of ginseng, increases endothelial nitric oxide synthase (eNOS) leading to increase nitric oxide (NO) production in endothelial cell (Leung K W, Cheng Y K, Mak N K. Signaling pathway of ginsenoside-Rg1 leading to nitric oxide production in endothelial cells. FEBS Lett. 2006; 580:3211-6). Ginsenosides can enhance Nitric Oxide production by induction of iNOS in addition to its direct effect on endothelial cells by increasing intracellular Ca2+ concentration (Li Z, Niwa Y, Sakamoto S. Induction of inducible nitric oxide synthase by ginsenosides in cultured porcine endothelial cells. Life Sci. 2000; 67:2983-9). Ginsenosides Rg3 is a potent inhibitor of vascular smooth muscle tone and that this effect seems to be due to an inhibition of Ca2+ influx and stimulation of K+ efflux, possibly via activation of tetraethylammonium-sensitive K+ channels (Kim N D, Kang S Y, Kim M J. The ginsenoside Rg3 evokes endothelium-independent relaxation in rat aortic rings: role of K+ channels. Eur J. Pharmacol. 1999; 367:51-7). A clinical randomized controlled trial showed that Korean ginseng can improve the vascular endothelial dysfunction in patients with hypertension possibly through increasing synthesis of nitric oxide (Sung J, Han K H, Zo J H. Effects of red ginseng upon vascular endothelial function in patients with essential hypertension. Am J Chin Med. 2000; 28:205-16). Ginsenoside Re can significantly inhibit cardiomyocyte apoptosis by inhibiting expression of pro-apoptotic Bax gene and raising the ratio of Bcl-2/Bax (Liu Z, Li Z, Liu X. Effect of ginsenoside Re on cardiomyocyte apoptosis and expression of Bcl-2/Bax gene after ischemia and reperfusion in rats. J Huazhong Univ Sci Technolog Med Sci. 2002; 22:305-9). Ginseng has a good effect on patients with congestive heart failure (Dai X, Zhou Y, Yu X. Effect of ginseng injection in treating congestive heart failure and its influence on thyroid hormones. Zhongguo Zhong Xi Yi Jie He Za Zhi. 1999; 19:209-11). A clinical randomized controlled trial showed that Ginseng and digoxin had synergism for treatment of congestive heart failure, and Ginseng was an effective and safe adjuvant without any side effects (Ding D Z, Shen T K, Cui Y Z. Effects of red ginseng on the congestive heart failure and its mechanism. Zhongguo Zhong Xi Yi Jie He Za Zhi. 1995; 15:325-7). The incorporation of this phytomedicine provides at least 86 active principles in a single therapeutic.

Pfaffia paniculata (Suma, Brazilian Ginseng, Pfaffia, Para Tudo, Corango-acu; also Hebanthe paniculata, Gomphrena paniculata, G. eriantha, Iresine erianthos, I. paniculata, I. tenuis, P. eriantha, Xeraea paniculata) contains active glycosides (beta-ecdysone and three ecdysteroids), six different pfaffic acids, phytosterols (sitosterol and estigmasterol). Pfaffia also contains saponins and 19 different amino acids, minerals, vitamins and pantoneic acid. Its germanium content probably accounts for its properties as an oxygenator at the cellular level and its high iron content may account for its traditional use for anemia. Ecdysteroids increased the muscle mass and normalize NADH dehydrogenase activity, enzyme which catalizes NADH electron transfer to the ubiquinone in the oxidative phosphorylation processes at the mitochondrial level, contributing to buildup the electrochemical potential used to produce ATP. It also normalizes the succinate dehydrogenase activity, enzyme which acts in the tricarboxilic acid cycle, which translates in ATP synthesis and patient energy level increases [Tashmukhamedova M A, Almatov K T, Syrov V N. Comparative study of the effect of ecdysterone, turkesterone and nerobol on the function of rat liver mitochondria in experimental diabetes. Vopr Med Khim. 1986; 32:24-8]. The incorporation of this phytomedicine provides 44 active principles in a single therapeutic.

Rhaponticum carthamoides (Leuzea carthamoides or Maral Root) contains a mixture of compounds called ‘levseins’. Levseins represents a complex of more than 10 ecdysterones including 20-beta-ecdysterone, makisterone C, 24-dehydromakisterone A, carthamosterone, polypodyne B and ajugasterone C. Researchers extracted and purified various ecdysteroids from Rhaponticum and found that the ecdysteroids increased the muscle mass in a dose-dependent manner, with the rate of increase proportional to the ecdysteroids content. Ecdysteroids normalize NADH dehydrogenase activity, enzyme which catalyses NADH electron transfer to the ubiquinone in the oxidative phosphorylation processes at the mitochondrial level, contributing to buildup the electrochemical potential used to produce ATP. It also normalizes the succinate dehydrogenase activity, enzyme which acts in the tricarboxilic acid cycle, which translates in ATP synthesis and patient energy level increases [Tashmukhamedova M A, Almatov K T, Syrov V N. Comparative study of the effect of ecdysterone, turkesterone and nerobol on the function of rat liver mitochondria in experimental diabetes. Vopr Med Khim. 1986; 32:24-8]. Incorporation of this phytomedicine in a composition provides at least 10 active principles in a single therapeutic.

Rhodiola rosea (Golden Root, Roseroot) consists mainly of phenylpropanoids (rosavin, rosin, rosarin (specific to R. rosea), phenylethanol derivatives (salidroside, rhodioloside, tyrosol), flavanoids (catechins, proanthocyanidins, rodiolin, rodionin, rodiosin, acetylrodalgin, tricin), monoterpenes (rosiridol, rosaridin), triterpenes (daucosterol, beta-sitosterol), and phenolic acids (chlorogenic and hydroxycinnamic, gallic acids). It also contains organic acids (gallic, caffeic, and chlorogenic acids) and p-Tyrosol. There are many species of Rhodiola, but it appears that the rosavins are unique to R. Rosea, and it is the preferred species. Rhodiola increases energy levels because it activates ATP synthesis and re-synthesis in mitochondria, stimulating reparative processes after intense exercise. It has properties of vasodilatation by activation of mu-opiate receptors in the cardiovascular system, and it is a hypolipidemic, diminishing cholesterol and triglyceride levels. Incorporation of this phytomedicine provides at least 28 active principles in a single therapeutic.

### Bio-Intelligence Modulators.—

Andrographis paniculata (King of Bitters, Kalmegh, Quasabhuva, The Creat and Kirayat) Primary active principles associated with Andrographis (AG) are: flavonoids, glucosides and diterpenic lactones (andrographolides). Vasodilator, hypotensive: Andrographis Paniculata increases nitric oxide, cyclic guanosine monophosphate and activity of superoxide dismutase, while decreases lipid peroxide and endothelin. Andrographis possesses the effects of antioxidation, preserving endothelial function, and maintaining the balance of nitric oxide/endothelin (Wang H W, Zhao H Y, Xiang S Q. Effects of Andrographis paniculata component on nitric oxide, endothelin and lipid peroxidation in experimental atherosclerotic rabbits. Zhongguo Zhong Xi Yi Jie He Za Zhi. 1997; 17:547-9).

14-deoxy-11,12-didehydroandrographolide (DDA) from Andrographis paniculata was elucidated in anaesthetized Sprague-Dawley (SD) rats and isolated rat right atria. DDA produced significant falls in mean arterial blood pressure and heart rate. The hypotensive action of DDA seems to work via adrenoceptors, autonomic ganglia receptor and angiotensin-converting enzyme. DDA caused negative chronotropic action and antagonized isoproterenol-induced positive chronotropic actions. These results further supported the bradycardia-inducing and beta-adrenoceptor antagonistic properties of DDA in vivo (Zhang C, Kuroyangi M, Tan B K. Cardiovascular activity of 14-deoxy-11,12-didehydroandrographolide in the anaesthetized rat and isolated right atria. Pharmacol Res. 1998; 38:413-7). Andrographis paniculata, its three semi-purified ethyl acetate (FA), n-butanol (FB) and aqueous (FC) fractions, as well as andrographolide, a major plant constituent, were elucidated in anaesthetized Sprague-Dawley (SD) rats. FB and FC produced a significant fall in mean arterial blood pressure without significant decrease in heart rate. The hypotensive action of FB seems to work via alpha-adrenoceptors, autonomic ganglion and histaminergic receptors (Zhang C Y, Tan B K. Mechanisms of cardiovascular activity of Andrographis paniculata in the anaesthetized rat. J. Ethnopharmacol. 1997; 56:97-101). Andrographolide from Andrographis paniculata has antithrombotic, hypotensive and antiatherosclerotic effects. It could be shown that andrographolide inhibits PAF-induced human blood platelet aggregation (Amroyan E, Gabrielian E, Panossian A. Inhibitory effect of andrographolide from Andrographis paniculata on PAF-induced platelet aggregation. Phytomedicine. 1999; 6:27-31).

Astragalus membranaceus (Huang-Qi, Huangqi) This plant contains three main types of active principles: isoflavones, which act as anti-oxidants; astragalans which act as immune-stimulants and anti-inflammatory; and astragalosides which increase arterial and coronary flow, and improve heart function. Astragaloside IV is the major active constituent of Astragalus membranaceus, which has been widely used for the treatment of cardiovascular diseases in China. Astragaloside IV significantly reduces infarct size in vivo. Also improves postischemic heart function and ameliorates reperfusion arrhythmias in rat hearts in vitro. The cardioprotection of astragaloside IV is accompanied by a significant increase in coronary flow. Astragaloside increases nitric oxide activating the nitric oxide synthase, and myocardial antioxidative enzyme superoxide dismutase activity. These data suggest the potential roles of antioxidative and nitric oxide-inducing properties of astragaloside IV in its protection from myocardial ischemia (Zhang W D, Chen H, Zhang C. Astragaloside IV from Astragalus membranaceus shows cardioprotection during myocardial ischemia in vivo and in vitro. Planta Med. 2006; 72:4-8). The results of one study indicate that Astragalus membranaceus can produce vascular relaxation. The mechanism may include the inhibition of intracellular calcium ions release by the 1,4,5-triphosphate inositol-receptor-dependent pathway in vascular smooth muscle cells (Zhang B Q, Hu S J, Shan Q X. Relaxant effect of Astragalus membranaceus on smooth muscle cells of rat thoracic aorta. Zhejiang Da Xue Xue Bao Yi Xue Ban. 2005; 34:65-8, 72). The effects of different components isolated from A. membranaceus on protecting the cardiac function in the process of ischemia reperfusion may be related to the mechanism of improving energy metabolism, scavenging the oxygen free radicals and inhibiting the production of free radicals in the ischemic myocardium (Zhou J Y, Fan Y, Kong J L. Effects of components isolated from Astragalus membranaceus Bunge on cardiac function injured by myocardial ischemia reperfusion in rats. Zhongguo Zhong Yao Za Zhi. 2000; 25:300-2). Astragalus has therapeutic effects on sodium and water retention in heart failure, the mechanisms of which might be the improvement of cardiac and renal functions, partly correction of abnormal mRNA expressions of AVP system and AQP2, and amelioration of blunted renal response to ANP (Ma J, Peng A, Lin S. Mechanisms of the therapeutic effect of astragalus membranaceus on sodium and water retention in experimental heart failure. Chin Med J (Engl). 1998; 111:17-23).

Echinacea angustifolia or purpurea (Black Sampson, Purple Coneflower, Rudbeckia, Missouri Snakeroot, Red Sunflower) contains alkaloids (Isotussilagine, tussilagine), amides (echinacein, isobutylamides), carbohydrates (echinacin, polysaccharides (heteroxylan and arabinogalactan), inulin, fructose, glucose, pentose), glycosides (echinacoside), terpenoids (Germacrane), Cichoric acid, betaine, methyl-para-hydroxycinnamate, vanillin, phytosterols, and volatile oils.

Echinacea has been the subject of hundreds of clinical and scientific studies which have primarily used an extract of the root and aerial portions of the botanical. The rich content of polysaccharides, sesquiterpene esters and phytosterols in Echinacea are what make it an anti-inflammatory plant. This component of Echinacea also has cortisone-like actions which can help control the inflammatory reactions. Echinacea's synergistic anti-oxidative properties have also been recently documented by Dalby-Brown et al, 2005. The incorporation of this phytomedicine into compositions provides at least 70 active principles in a single therapeutic.

Ganoderma lucidum (Reishi, also LingZhi, G. tsugae, G. valesiacum, G. oregonense, G. resinaceum, G. pfezfferi, G. oerstedli, and G. ahmadii) is an edible fungus containing bitter triterpenoids (ganoderic acid), β-D-glucan, coumarins, alkaloids and ergosterols. The main active principles of this mushroom are sterols and beta-proteoglucans which bestow anti-inflammatory properties. Ganoderma total sterols significantly reduce malondialdehyde content and reactive oxygen species production and increases superoxide dismutase activity; furthermore, the translocation of nuclear factor-kappa B and the production of interleukin-1beta and tumor necrosis factor alpha induced by hypoxia/reoxygenation is blocked, suggesting that Ganoderma total sterols might be useful in treating hypoxia/reoxygenation-induced oxidative stress and inflammatory responses. Superoxide dismutase might play a critical role in the effect of Ganoderma against hypoxia/reoxygenation injury. In addition, Ganoderma component GS-1 significantly attenuated the formation of reactive oxygen species (Zhao H B, Wang S Z, He Q H. Ganoderma total sterol (GS) and GS1 protect rat cerebral cortical neurons from hypoxia/reoxygenation injury. Life Sci. 2005; 76:1027-37). The amino-polysaccharide fraction from Ganoderma lucidum protects against oxidative damage induced by reactive oxygen species. It also significantly inhibits lipid peroxidation and shows inactivation of hydroxyl radicals and superoxide anions (Lee J M, Kwon H, Jeong H. Inhibition of lipid peroxidation and oxidative DNA damage by Ganoderma lucidum. Phytother Res. 2001; 15:245-9). It has vasodilator effect and is useful in the treatment of angina. It is hypolipidemic and anti-atherotic. Ganoderma lucidum was shown to have antioxidative effect against heart toxicity.

It exhibited a dose-dependent antioxidative effect on lipid peroxidation and superoxide scavenging activity in heart. The antioxidative activity may therefore contribute to the cardioprotective effect of Ganoderma lucidum, and may therefore protect the heart from superoxide induced damage (Wong K L, Chao H H, Chan P. Antioxidant activity of Ganoderma lucidum in acute ethanol-induced heart toxicity. Phytother Res. 2004; 18:1024-6). Lingzhi is a popular Chinese herb with an impressive array of reputed health benefits, including antioxidant properties. However, these require scientific validation. A Clinical double-blinded Randomized Placebo-Controlled Trial showed that Ganoderma lucidum increases plasma antioxidant capacity, fasting plasma lipid alpha-tocopherol concentration and urine antioxidant capacity. Fasting plasma ascorbic acid and total alpha-tocopherol concentrations and erythrocyte SOD and GPx activities increased slightly. Plasma lipids and uric acid tended to decrease. These results indicate that Lingzhi intake causes an acute increase in plasma antioxidant capacity. The pattern of biomarker response indicated benefit in terms of antioxidant status and coronary heart disease risk (Wachtel-Galor S, Szeto Y T, Tomlinson B. Ganoderma lucidum (‘Lingzhi’); acute and short-term biomarker response to supplementation. Int J Food Sci Nutr. 2004; 55:75-83). The incorporation of this phytomedicine into compositions provides at least 32 active principles in a single therapeutic.

Grifola frondosa (Maitake, Dancing Mushroom; also G. sordulenta, Polyporus umbellatus and Meripilus giganteus) contains the primary polysaccharide, β-D-glucan in the 1.3 and 1.6 forms. It also contains alpha glucan, lipids, phospholipids, and ergosterol. β-D-glucan is recognized as an effective immuno-stimulator. Arterial Hypertension: Maitake powder has shown in animal tests to decrease the arterial pressure and to prevent its increase. Feeding rats with Maitake powder (5% of the diet) during 9 weeks significantly reduced the arterial tension. A similar feeding procedure during 8 weeks, initiated when the mice were 10 weeks old and had established arterial hypertension, was also successful. Maitake prevents the development of hypertension and also reduces elevated tension. Cholesterol and Triglycerides: Various studies have examined the effects Maitake has on seric lipids, including cholesterol and triglycerides.

In a study published in 1988, hypertense rats were fed with a mixture of 5% Maitake powder which significantly reduced the levels of VLDL and total cholesterol. Another study done on rats fed with a diet of high cholesterol contents, together with 20% of Maitake powder showed that Maitake inhibited the hepatic accumulation of fats and produced a reduction of total cholesterol. In another study with similar rules, the rats fed with Maitake experienced a significant and prolonged reduction of cholesterol and seric triglycerides, maintaining similar levels of HDL cholesterol. Maitake has some complex B vitamins, ergosterol/provitamin D2, magnesium, potash, calcium, unsaturated fat acids, phosphatidilsesrine and other phospholipids and proteins. Although it does not contain vitamins A or C, there have been identified some substances with chemical properties similar to ascorbic acid. Because Maitake is rich in fibers and low in calories and fats, it has been mentioned as a natural product to control overweight. Animal studies have shown that the daily supply of Maitake may inhibit weight gain. When rats were fed with dry Maitake powder, 20% in weight of a diet high in cholesterol, an increase in weight and corporal fats was significantly inhibited. A similar rule showed an improvement of the lipid metabolism in rats fed with Maitake. At the end of the study, those animals had 24.9% less weight than the control rats. The tests done on hypertense rats fed with Maitake also showed an inhibiting effect on weight gain. In a clinical study conducted on 30 overweight patients, investigators offered daily pills with an equivalent of 200 grams of fresh Maitake during two months. All patients lost weight without any special diet. The average weight loss was 7-13 pounds and one of them lost 26.4 pounds. Incorporation of Grifola provides at least 6 active ingredients for therapeutic use.

### Organizational Improvers.—

Coenzyme Q10 (CoQ10), also known as ubiquinone or ubiquinol, is a biologically active quinone with an isoprenoid side chain, related in structure to vitamin K and vitamin E. CoQ is found in the membranes of endoplasmic reticulum, peroxisomes, lysosomes, vesicles and notably the inner membrane of the mitochondrion where it is an important part of the electron transport chain; there it passes reducing equivalents to acceptors such as Coenzyme Q—cytochrome c reductase: CoQH₂+2Fe⁺³-cytochrome c→CoQ+2Fe⁺²-cytochrome c CoQ is also essential in the formation of the apoptosome along with other adapter proteins. The loss of trophic factors activates pro-apoptotic enzymes, causing the breakdown of mitochondria. Because of its ability to transfer electrons and therefore act as an antioxidant, Coenzyme Q has become a valued dietary supplement. Young people are able to make Q10 from the lower numbered ubiquinones such as Q6 or Q8. The sick and elderly may not be able to make enough, thus Q10 becomes essential later in life or in illness. Supplementation of Coenzyme Q10 is a common component of the ‘mito cocktail’ used to treat mitochondrial disorders and other metabolic disorders. Recent studies have shown that the antioxidant properties of Coenzyme Q10 benefit the body. A double-blind and double-crossover trial conducted by administering CoQ 10 and a matching placebo orally to two groups of patients having class III or IV cardiomiopathy (classification according to criteria of the New York Heart Association). Group A received CoQ10 and then placebo; group B received placebo and then CoQ10. For group A, significant increases in cardiac function occurred during CoQ10 treatment and then decreased during crossover to placebo. For group B, there was no change in cardiac function during placebo treatment. These patients, steadily worsening and expected to die within 2 years under conventional therapy, generally showed an extraordinary clinical improvement, indicating that CoQ10 therapy might extend the lives of such patients. This improvement could be due to correction of a myocardial deficiency of CoQ10 and to enhanced synthesis of CoQ10-requiring enzymes (Langsjoen P H, Vadhanavikit S, Folkers K. Response of patients in classes III and IV of cardiomyopathy to therapy in a blind and crossover trial with coenzyme Q10. Proc Natl Acad Sci USA. 1985; 82:4240-4).

Coleus forskohlii BRIQ (Lamiaceae) Active constituents: The labdane diterpene forskolin, derived from the root of the plant, is the primary constituent of clinical interest in Coleus forskohlii. It was discovered by Western scientists in 1974 and was initially referred to as coleonol. Since that time, as other coleonols and diterpenoids have been identified, the name was changed to forskolin. Forskolin is responsible for virtually all pharmacological activities attributed to Coleus forskohlii; extracts of this constituent have been used in nearly all existing studies.

There is evidence, however, that other plant constituents, such as volatile oils and other diterpenoids and coleonols, may contribute to the pharmacological activity and absorption of forskolin. Detailed analysis reveals approximately 20 constituents in various parts of the plant, but forskolin and other coleonols are present only in the root portion. Forskolin's primary mode of action is to increase cyclic adenosine monophosphate (cAMP) and cAMP-mediated functions, via activation of the enzyme adenylate cyclase. Forskolin has been shown to increase cAMP formation in all eukaryotic cells, without hormonal activation of adenylate cyclase. Forskolin's potentiation of cAMP in turn lowers blood pressure, inhibits platelet aggregation, promotes vasodilation, and stimulates lipolysis in fat cells. Forskolin also has cardiotonic effects with a positive inotropic action on cardiac tissue via increased cAMP levels. In addition to its cAMP-stimulating activity, forskolin inhibits the binding of platelet-activating factor (PAF), independently of cAMP formation. This may be a result of forskolin's direct effect on PAF or via interference with PAF binding to receptor sites. Both animal and clinical studies demonstrate forskolin significantly lowers blood pressure via relaxation of vascular smooth muscle. In a small study of seven patients with dilated cardiomyopathy, forskolin significantly reduced diastolic blood pressure (17%) without increasing myocardial oxygen consumption; left ventricular function also improved. In a similar study, forskolin given to dilated cardiomyopathy patients resulted in decreased vascular resistance and a 19-percent improvement in left ventricle contractility. Heart rate increased an average of 16 percent in study patients. Subjects also exhibited a 20-percent reduction in arterial pressure accompanied by symptomatic flush. Forskolin's ability to inhibit platelet aggregation is of additional benefit in cardiovascular disease. Forskolin provides at least 20 active principles to the formulation.

Crataegus oxyacantha (Hawthorn, also C. monogyna) contains mainly flavonoids (such as flavonoglycosyls, hyperoside, rutin, flavonol, kaempferol and quercetin) and oligomeric procyanidins (1-epicatechol), which relax arterial and decrease peripheral vascular resistance. Crataegus also contains amines (phenyletylamine, tyramine, O-methoxyphenethylamine), flavone (apigenin, luteolin) derivatives, vitexin glycosides, tannins, saponins, and cyanogenetic glycosides.

Vasodilator: Crataegus extract induces an endothelium-dependent, NO-mediated vasorelaxation via eNOS phosphorylation (Brixius K, Willms S, Napp A. Crataegus Special Extract WS((R)) 1442 Induces an Endothelium-Dependent, NO-mediated Vasorelaxation via eNOS-Phosphorylation at Serine 1177. Cardiovasc Drugs Ther. 2006 Jun. 21). Procyanadins of Crataegus caused endothelium-dependent relaxation which was associated with the production of cyclic GMP. Procyanidins of Crataegus may be responsible for the endothelium-dependent nitric oxide-mediated vascular relaxation, possibly via activation of tetraethylammonium-sensitive K+ channels (Kim S H, Kang K W, Kim K W. Procyanidins in crataegus extract evoke endothelium-dependent vasorelaxation in rat aorta. Life Sci. 2000; 67:121-31). Results observed suggest that Hawthorn flavonoids protect endothelial cell from hypoxia partly through its regulative effect on NO and calcium ion levels (Lan W J, Ge Y K, Zheng X X. Regulative effects of hawthorn leave flavonoids on cytotoxicity, NO and Ca2+ in human endothelial cells. Space Med Med Eng (Beijing). 2005; 18:157-60). Flavonoids and proanthocyanidins from Crataegus oxyacantha/C. monogyna demonstrated inhibitory activity of Angiotensin Converting Enzyme (ACE). (Lacaille-Dubois, Franck U, Wagner H. Search for potential angiotensin converting enzyme (ACE)-inhibitors from plants. Phytomedicine. 2001; 8:47-52). One randomized controlled trial showed the hypotensive effect of hawthorn in patients with diabetes taking medication. (Walker A F, Marakis G, Simpson E. Hypotensive effects of hawthorn for patients with diabetes taking prescription drugs: a randomized controlled trial. Br J Gen Pract. 2006; 56:437-43). Cardiotonic: The results of recent studies provide evidence that extract of hawthorn (Crataegus sp.) may provide benefits in left ventricular systolic dysfunction. The authors present a number of studies in which the influence of this herbal drug on contractility of impaired myocardium has been proved. This therapy was well tolerated and no interactions with the other compounds for heart failure were reported (Rechcinski T, Kurpesa M. Oligomeric procyanidins from hawthorn extract as supplementary therapy in patients with left ventricle systolic dysfunction. Przegl Lek. 2005; 62:243-4). The data of one multicenter study showed a clear benefit for patients with heart failure stage NYHA II treated with WS 1442.

The single or add-on administration in addition to a chemical-synthetic medication resulted in objective improvements at comparable costs (Habs M. Prospective, comparative cohort studies and their contribution to the benefit assessments of therapeutic options: heart failure treatment with and without Hawthorn special extract WS 1442. Forsch Komplementarmed Klass Naturheilkd. 2004; 11:36-9). In Germany, extracts from Crataegus spp. are approved drugs for the treatment of mild forms of heart insufficiency. Besides cardiotonic effects these herbal remedies have been shown to possess cardioprotective properties. Oral administration of extracts from Crataegus spp attenuated the elevation of the ST-segment in the ECG, diminished the incidence of ventricular fibrillations and reduced the mortality rate. Furthermore, the area of myocardial infarction within the ischemic zone was significantly smaller. These pharmacological effects are accounted for by the combined antioxidative, leukocyte elastase inhibiting and endothelial nitric oxide (NO) synthesis enhancing properties of extracts from Crataegus spp (Veveris M, Koch E, Chatterjee S S. Crataegus special extract WS 1442 improves cardiac function and reduces infarct size in a rat model of prolonged coronary ischemia and reperfusion. Life Sci. 2004; 74:1945-55). A placebo controlled, randomised, parallel group, multicentre trial shows the efficacy and safety of a standardised extract of fresh berries of Crataegus in patients with cardiac failure NYHA class II (Degenring F H, Suter A, Weber M. A randomised double blind placebo controlled clinical trial of a standardised extract of fresh Crataegus berries (Crataegisan) in the treatment of patients with congestive heart failure NYHA II. Phytomedicine. 2003; 10:363-9). One meta-analysis of thirteen trials with Crataegus extracts for the treatment of chronic heart failure showed that treatment with hawthorn extract was more beneficial than placebo. The pressure-heart rate product also showed a beneficial decrease. Symptoms such as dyspnea and fatigue improved significantly with hawthorn treatment. These results suggest that there is a significant benefit from hawthorn extract as an adjunctive treatment for chronic heart failure. (Pittler M H, Schmidt K, Ernst E. Hawthorn extract for treating chronic heart failure: meta-analysis of randomized trials. Am J. Med. 2003; 114:665-74).

One clinical, randomized controlled trial showed a reduction in the resting diastolic blood pressure with the hawthorn extract (Walker A F, Marakis G, Morris A P. Promising hypotensive effect of hawthorn extract: a randomized double-blind pilot study of mild, essential hypertension. Phytother Res. 2002; 16:48-54). A randomised, placebo-controlled, double-blind clinical study in patients suffering from congestive heart failure NYHA class II showed that the exercise tolerance increased. The difference of the double product (heart rate×systolic blood pressure×10(−2)) decreased. Crataegus was safe and well tolerated. The data show that Crataegus extract is clinically effective in patients with congestive heart failure corresponding to NYHA class II (Zapfe jun G. Clinical efficacy of crataegus extract WS 1442 in congestive heart failure NYHA class II. Phytomedicine. 2001; 8:262-6). Extracts from Crataegus exert direct positive inotropic effects. This study was designed to investigate the mode of its inotropic action in human myocardium from patients with congestive heart failure (left ventricular myocardium from explanted hearts; NYHA IV. Crataegus extracts significantly increased the force of contraction and improved the frequency-dependent force generation even in failing human myocardium. It also increased both the Ca2+-transient and force generation. These findings suggest a pharmacologic mechanism of Crataegus extracts similar to the cAMP-independent positive inotropic action of cardiac glycosides. In addition, Crataegus improves the force-frequency relation in failing human myocardium (Schwinger R H, Pietsch M, Frank K. Crataegus special extract WS 1442 increases force of contraction in human myocardium cAMP-independently. J Cardiovasc Pharmacol. 2000; 35:700-7). The efficacy and tolerance of the standardized hawthorn (crataegus) extract were tested in a multicenter utilization observational study on 1,011 patients with cardiac insufficiency stage NYHA II. A significant improvement in clinical symptoms (reduced performance in the exercise tolerance test, fatigue, palpitation and exercise dyspnea) was observed. Ankle edema and nocturia disappeared by 83%, and by half of the patients respectively manifesting these symptoms before treatment. The improvement and economization of cardiac performance were additionally shown by a reduction in blood pressure, an increased maximal exercise tolerance and a reduction in the difference in the pressure/heart rate product (PHRP).

The positive effects of Crataegus extract were further demonstrated by an improved ejection fraction and an increased percentile shortening fraction measured using M-mode echocardiography. The stabilizing effect of the hawthorn extract on the heart rate was shown by a slower rest pulse, as well as by an increase in the number of day and night normorhythmic patients, as documented by long-term ECG. The reduction in the number of patients showing ST depressions, arrhythmias and ventricular extrasystoles at the maximum exercise level is regarded as an indication for an improved myocardial perfusion. Almost ⅔ of the patients felt better or much better following the 24 weeks of treatment. More than ¾ of the participating physicians noted a good or a very good efficacy, and 98.7% noted a good or a very good tolerance. High-dose hawthorn therapy is an efficient, well-tolerated and easily regulated therapeutic alternative for patients suffering from cardiac insufficiency stage NYHA II. (Tauchert M, Gildor A, Lipinski J. High-dose Crataegus extract WS 1442 in the treatment of NYHA stage II heart failure. Herz. 1999; 24:465-74).

Croton lechleri (Dragon's blood, Sangre de Grado, Sangre de Agua; also C. draconoides, C. palanostigma, C. erythrochilus C. salutaris, and C. gossypifolius) produces a distinctive red exudate from its trunk containing a considerable amount of secondary plant metabolites, the majority of which are hydrolyzing flavonoids, proanthocyanidins (mainly catechin, epicatechin, gallocatechin and/or galloepicatechin), as well as taspine. Other components include the dihydrobenzofuran lignan, six simple phenols and their derivatives, three steroids, non-saturated fatty acids, diterpenoids (hardwickiic acid, bincatriol, crolechinol, crolechinic acid, coberine A, coberine B) and diterpenoids. These active principles explain its anti-oxidant and anti-inflamatory activities. Croton lechleri sap possesses significant antioxidant activity against the oxidative damages (Lopes M I, Saffi J, Echeverrigaray S. Mutagenic and antioxidant activities of Croton lechleri sap in biological systems. J. Ethnopharmacol. 2004; 95:437-45). Croton lechleri was highly effective in reducing oxidation of DNA (Desmarchelier C, Witting Schaus F, Coussio J. Effects of Sangre de Drago from Croton lechleri Muell.-Arg. on the production of active oxygen radicals. J. Ethnopharmacol. 1997; 58:103-8).

Incorporation of this phytomedicine into a composition provides at least 23 active principles in a single therapeutic.

Gynostemma pentaphyllum: Contains Gypenosides (triterpenoid saponins); dammarane glycosides, gylongiposide I, allantion, vitexin, Gynosides A-E (ocotillone-type saponins); rutin, ombuoside and malonic acid (flavonoids). The results of one study showed that G. pentaphyllum produced a protective effect against coronaryspasm, arrhythmias and pressor responses. Extract also increased the dose of ouabain required to cause ventricular tachyarrhythmias and lethality. The extract reversed ouabain-induced persistent ventricular tachycardia and restored sinus rhythm. (Circosta C, De Pasquale R, Occhiuto F. Cardiovascular effects of the aqueous extract of Gynostemma pentaphyllum Makino. Phytomedicine. 2005; 12:638-43). Gypenosides from Gynostemma pentaphyllum are reported to be effective in the treatment of cardiovascular diseases; these active principles elicit vasorelaxation through the direct release of endothelium-derived nitric oxide (Tanner M A, Bu X, Steimle J A. The direct release of nitric oxide by gypenosides derived from the herb Gynostemma pentaphyllum. Nitric Oxide. 1999; 3:359-65). Gynostemma pentaphyllum inhibit significantly the platelet aggregation, accelerate obviously the disaggregation and inhibit effectively the experimental thrombosis. This herb could decrease the activity of multiple coagulation factors. This study revealed that GP is an antithrombotic agent affecting the links of thrombotic chain. (Tan H, Liu Z L, Liu M J. Antithrombotic effect of Gynostemma pentaphyllum. Zhongguo Zhong Xi Yi Jie He Za Zhi. 1993; 13:278-80, 261). Gypenosides of Gynostemma pentaphyllum inhibit lipid peroxidation in vascular endothelial cells. It also protected biomembranes from oxidative injury by reversing the decreased membrane fluidity of mitochondria, increasing mitochondrial enzyme activity in vascular endothelial cells and decreasing intracellular lactate dehydrogenase leakage from these cells. The extensive antioxidant effect of GP is of value to the prevention and treatment of various diseases such as atherosclerosis (Li L, Jiao L, Lau B H. Protective effect of gypenosides against oxidative stress in phagocytes, vascular endothelial cells and liver microsomes. Cancer Biother. 1993; 8:263-72). Provides at least 30 active principles.

Radix Polygalae (Polygala tenuifolia, Polygala sibirica, Chinese Senega, Polygala fallax, Polygala caudata, Polygala paniculata). Active principles: Xanthones, Oleanane-type Triterpenoid saponins (Polygalasaponins, Onjisaponins, Reiniosides, Tenuifolin, Tenuidin, Tenuigenin, Presenegenin, Senegenin, Senegasaponins and senegins); Phytosterols (Daucosterol), oligosaccharide esters (Senegoses A-E, Tenuifoliose Q); phenolic compounds (polygalolides A-B) and fatty acids (oleic acid, linoleic acid, palmitic acid, eicosenoic acid and stearic acid). Xanthones of Polygala show different anti-oxidation activities: scavenger activity of the reactive oxygen free radicals (Lin L L, Huang F, Chen S B. Chemical constituents in roots of Polygala fallax and their anti-oxidation activities in vitro. Zhongguo Zhong Yao Za Zhi. 2005; 30:827-30) (Lin L L, Huang F, Chen S B. Xanthones from the roots of Polygala caudata and their antioxidation and vasodilatation activities in vitro. Planta Med. 2005; 71:372-5). Radix polygalae provides 31 active principles.

Tabebuia avellanedae (Pau d'arco, Ipê, Lapacho, Tahuari, Taheebo, Trumpet Tree, Tabebuia Ipê, Tajy; also T. ipe, T. nicaraguensis, T. schunkeuigoi, T. serratifolia, T. altissima, T. palmeri, T. impetiginosa, T. heptaphylla, Gelseminum avellanedae, Handroanthus avellanedae, H. impetiginosus, Tecoma adenophylla, Tec. avellanedae, Tec. eximia, Tec. impetiginosa, Tec. integra, Tec. ipe) extracts contain diverse quinone derivatives and a small quantity of benzenoids and flavonoids, including beta-lapachone, xyloidone, tabebuin, quercetin, tecomine, and steroidal saponins. One important ingredient is lapachol, a derivative of which was patented in 1975. It has anti-inflammatory effects. The antioxidant activity of Tabebuia was evaluated. The extract exhibited a potent inhibitory effect on the formation of conjugated diene hydroperoxides; inhibited the oxidation of hexanal. The antioxidative activity was comparable with that of the well-known antioxidants, alpha-tocopherol, and butylated hydroxytoluene (Park B S, Lee K G, Shibamoto T. Antioxidant activity and characterization of volatile constituents of Taheebo—Tabebuia impetiginosa Martius ex D C. J Agric Food Chem. 2003; 51:295-300). Extracts of Tabebuia showed marked and selective inhibition of platelet aggregation. These extracts also significantly suppressed arachidonic acid liberation in platelets.

Potently inhibited cell proliferation and DNA synthesis induced by platelet derived growth factor (PDGF)-BB, and inhibited the levels of phosphorylated extracellular signal regulated kinase (ERK1/2) mitogen activated protein kinase (MAPK) stimulated by PDGF-BB (Son D J, Lim Y, Park Y H. Inhibitory effects of Tabebuia impetiginosa inner bark extract on platelet aggregation and vascular smooth muscle cell proliferation through suppressions of arachidonic acid liberation and ERK1/2 MAPK activation. J. Ethnopharmacol. 2006 Apr. 28). Results of one study indicate that beta-lapachone is capable of inhibiting expression and function of inducible nitric oxide synthase in aortic rings. It is considered that beta-lapachone can be developed as a potential anti-inflammatory agent in the future (Liu S H, Tzeng H P, Kuo M L. Inhibition of inducible nitric oxide synthase by beta-lapachone in rat alveolar macrophages and aorta. Br J Pharmacol. 1999; 126:746-50). Incorporation of Tabebuia provides at least 32 active principles in a single therapeutic.

Tribulus terrestris (Puncture Vine, Caltrop, Yellow Vine, bindy eye, bindii, bullhead, burnut, burra gokhroo, caltrop, calthrops, cat's head, common dubbeltjie, devil's thorn, devil's weed, doublegee, dubbeltje, goathead, gokshura, ground bur-nut, isiHoho, land caltrop, Maltese cross, Mexican sandbur, puncture vine, puncture weed, rose, small caltrops, tackweed, Texas sandbur, yellow vine and Goathead). The fruits and roots of Tribulus contain active principles such as: phytosterols, flavonoids, alkaloids, glucosides and steroidal saponins of the furostanol sub-class with a predominant amount of protodioscine (no less than 45%) which seems to be the principle that produces the clinical results. In one clinical study, 406 cases of coronary heart disease were treated with Tribulus terrestris saponins. The results showed that the total efficacious rate of remission angina pectoris was 82.3%. It was higher than the control group with a total effective rate of 67.2%. The total effective rate of ECG improvement (52.7%) was even higher than that of the control group (35.8%). It was shown that saponin of Tribulus terrestris has the action of dilating coronary artery and improving coronary circulation, and thus has better effects on improving ECG of myocardial ischemia. If taken for a long time, it has no adverse reaction on blood system and hepatic and renal functions. Neither does it have side effects.

It is one of the ideal medicines to treat angina pectoris (Wang B, Ma L, Liu T. 406 cases of angina pectoris in coronary heart disease treated with saponin of Tribulus terrestris. Zhong Xi Yi Jie He Za Zhi. 1990; 10:85-7). Tribulus terrestris possess significant antihypertensive activity, resulting from a direct arterial smooth muscle relaxation possibly involving nitric oxide release and membrane hyperpolarization (Phillips O A, Mathew K T, Oriowo M A. Antihypertensive and vasodilator effects of methanolic and aqueous extracts of Tribulus terrestris in rats. J. Ethnopharmacol. 2006; 104:351-5). A study designed to observe the effect of Tribuli saponins on left ventricular remodeling after acute myocardial infarction showed that: (1) Cardiac function: Tribulus saponins increased the cardiac fractional shortening and ejection fraction, and lowered the left ventricular end diastolic volume (LVEDV) and systolic volume (LVESV). (2) Cardiac structure: the left ventricular dimension end diastole (LVDd) and systole (LVDs) in the treated groups got lower (3) Ventricular weight index: the treated groups showed a decrease in the left ventricular weight index. Tribulus terrestris could attenuate the left ventricular remodeling after acute myocardial infarction, and improve cardiac function in the early phase after acute myocardial infarction, thus playing an important role in controlling morbidity and mortality of cardiac events and long-term prognosis (Guo Y, Yin H J, Shi D Z. Effects of Tribuli saponins on left ventricular remodeling after acute myocardial infarction in rats with hyperlipidemia. Chin J Integr Med. 2005; 11:142-6).

Vitamin E: Considerable epidemiologic data suggest that dietary consumption of vitamin E reduces the incidence of cardiovascular disease. Precise mechanisms are not clear, but emerging data indicate that vitamin E has numerous activities that may, in part, explain its effect on vascular disease. In particular, vitamin E enhances the bioactivity of nitric oxide, inhibits smooth muscle proliferation, and limits platelet aggregation. A common mechanism to account for these effects of vitamin E is the inhibition of protein kinase C stimulation. In the setting of atherosclerosis, inhibition of protein kinase C by vitamin E would be expected to maintain normal vascular homeostasis and reduce the clinical incidence of cardiovascular disease (Keaney J F Jr, Simon D I, Freedman J E. Vitamin E and vascular homeostasis: implications for atherosclerosis. FASEB J. 1999; 13:965-75).

A double-blind, placebo-controlled, randomized study in 2002 patients with angiographically proven coronary atherosclerosis concludes that alpha-tocopherol treatment substantially reduces the rate of non-fatal myocardial infarction (Stephens N G, Parsons A, Schofield P M. Randomised controlled trial of vitamin E in patients with coronary disease: Cambridge Heart Antioxidant Study (CHAOS). Lancet. 1996; 347:781-6). Impairment of endothelium-dependent vasodilation is associated with the initiation and development of atherosclerosis. Vasodilator prostanoids constitute a protective mechanism in maintaining normal vasomotor function. The vitamin E-induced increase in PGI(2) and PGE(2) production may contribute to its suggested beneficial effect in preserving endothelial function (Wu D, Liu L, Meydani M. Vitamin E increases production of vasodilator prostanoids in human aortic endothelial cells through opposing effects on cyclooxygenase-2 and phospholipase A2. J. Nutr. 2005; 135:1847-53). Vitamin E is protective against coronary heart disease (He M, Jiang C, Zheng H. Study on the relationship between the polymorphism of P22phox C242T, vitamin E and coronary heart disease. Wei Sheng Yan Jiu. 2004; 33:443-6). Vitamin E prevented LPC-induced endothelial dysfunction and preserved endothelial nitric oxide release, (b) vitamin E inhibited LPC-induced platelet activation (P-selectin expression) and leukocyte-platelet interaction, and (c) these mechanisms appeared to be at least partly mediated by suppression of the PKC in endothelial cells and platelets. The present findings may provide new insights into antiatherogenic mechanisms of vitamin E (Murohara T, Ikeda H, Katoh A. (Vitamin E inhibits lysophosphatidylcholine-induced endothelial dysfunction and platelet activation. Antioxid Redox Signal. 2002; 4:791-8). A clinical randomized controlled trial showed that hypercholesterolemia both increased circulating soluble cell adhesion molecule-1 and reduced nitric oxide metabolite concentrations. Vitamin E supplementation counteracts these alterations, thus representing a potential tool for endothelial protection in hypercholesterolemic patients.

### EXAMPLE 2

### Composition—Cardiac Disorders

A particularly preferred composition is shown in Table 1. Ratios reflect concentration of active ingredients over the natural state. Amounts provided are mg of extract. Obviously the amount should be increased where the strength is reduced and vice versa.

TABLE 1
Composition

| Active Agent | Ratio | Amount (mg) |
|---|---|---|
| Energy enhancers | | |
| Panax ginseng | 1:1 | 221 |
| Panax quinquefolius | 1:1 | 221 |
| Rhapontium carthamoides | 6:1 | 4 |
| Rhodiola rosea | 10:1 | 14 |
| Bio-lntelligence modulators | | |
| Andrographis paniculata | 10:1 | 44 |
| Astragalus membrenaceus | 10:1 | 44 |
| Echinacea | 10:1 | 44 |
| Ganoderma lucidum | 10:1 | 44 |
| Grifola frondosa | 10:1 | 44 |
| Organization improvers | | |
| Coenzyme Q10 | 1:1 | 8 |
| Coleus forskohlii | 1:1 | 71 |
| Crataegus oxycantha | 10:1 | 27 |
| Croton lechleri | 5:1 | 2 |
| Gynostemma pentaphyllum | 5:1 | 7 |
| Radix polygalae | 5:1 | 7 |
| Tabebuia avellanedae | 10:1 | 44 |
| Tribulus terrestris | 5:1 | 35 |
| Vitamin E | 1:1 | 18 |
| Total | | 900 |

### EXAMPLE 3

### A Clinical Study of the Formulation's Effectiveness and Tolerance

A retrospective, multicenter, descriptive three year long study was undertaken to evaluate the effects of the therapeutic formula object of the patent—and formulated under the precepts of Systemic Medicine—in 80 patients diagnosed with Congestive Cardiac Failure (CHF) in NYHA Class III and IV. The composition improved: dyspnea—at rest—in all patients (100%); dyspnea at exercise in 93.7% of the patients; orthopnea in 95.8%; peripheric edema in 93.4%; generalized edema in 100%; hepatomegaly in 83.3%, cough in 97.2%; tachycardia in 97.2%; urinary symptoms in 95.8%; hiporexia in 94.7% and fatigue in 94.8% of all patients. Additionally, Quality of Life in 93.7% of the population. Tolerance to the treatment was excellent (100%), no side effects were reported.

### EXAMPLE 4

### Principles for Selecting Synergistic Combinations

In order to explain the range of formulations encompassed by the invention, we have categorized beneficial plants and nutraceuticals into one of three groups, each of which should be present for synergistic effect. The classifications are: Energy, Bio-Intelligence and Organization. Plants and nutraceuticals classified under Energy are associated with ATP synthesis (such as the Krebs cycle, oxidative phosphorylation, beta-oxidation, etc.). Plants and nutraceuticals classified under Bio-Intelligence are those that regulate the neuroendocrine and immunological systems and cellular processes, thus controlling the interactions between the various systems in the body. Finally, plants and nutraceuticals classified under Organization are those that relate to the structure and function of specific organs. Combinations of plants and nutraceuticals from these three classification groups have synergistic effect because they address each necessary component of cellular and organic health—in effect they provide the triangle on which healing is fully supported.

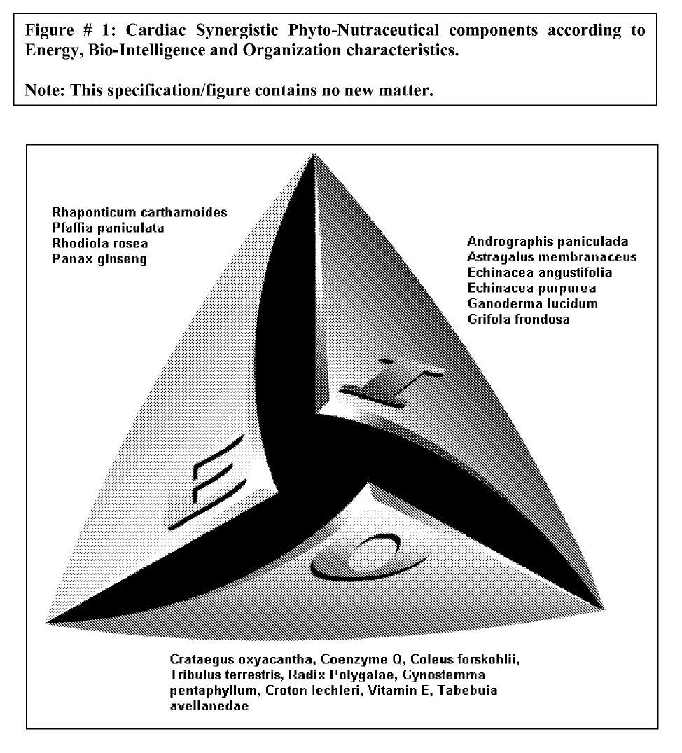

FIG. 1, depicts the components—plants and/or nutraceuticals—which enhance Energy (E), modulate Bio-Intelligence (I) and improve Organization (O) sides of the aforementioned health triangle. That is the components listed on the left hand view of FIG. 1, are the plants and/or nutraceuticals that enhance Energy. The plants and/or nutraceuticals in the right view are those that improve Organization. Finally, the plants and nutraceuticals at the bottom view of FIG. 1 modulate Bio-Intelligence.

An illustrative example of synergy in medicinal plants is an in vitro study that demonstrates how the activity of herbal Berberine alkaloids is strongly potentiated by the action of 5′-methoxyhydnocarpin (5′-MHC)—an active principle of another phytomedicine (denominated Hydnocarpus wightiana). It shows a strong increase of accumulation of berberine in the cells in the presence of 5′-MHC, indicating that this plant compound effectively disabled the bacterial resistance mechanism against the berberine antimicrobial, thus showing the synergy of both substances. Stermitz F R, et al., Synergy in a medicinal plant: antimicrobial action of berberine potentiated by 5′-methoxyhydnocarpin, a multidrug pump inhibitor. Proc Natl Acad Sci USA. 2000; 97:1433-7.

A further demonstration may be provided of synergistic effect on a molecular scale by studying the gene expression profile changes in response to various plant ingredients and combinations thereof. Experiments are already underway demonstrating the expression profile in response to the formulations. We will be aided in this work because researchers have already begun studying the expression profiles of various medicinal plants, thus providing a database of knowledge from which to build. E.g., Gohil, et al., mRNA Expression Profile of a Human Cancer Cell Line in Response to Ginkgo Biloba Extract: Induction of Antioxidant Response and the Golgi System, Free Radic Res. 2001; 33:831-849.

Finally there may be further presentation of gene expression results using microarray analysis to demonstrate the formulation's capability to provide gene modulation (upregulation or downregulation).

It may also be possible to add tests of plants' combinations for further demonstration of synergistic effects by using experimental models.

What is claimed is:

1) A phytoceutical composition, comprising plants or extracts or active ingredients derived from each of the following plants and nutraceuticals: Panax, Pfaffia, Rhapontium, Rhodiola, Andrographis, Astragalus, Echinacea, Ganoderma, Grifola, Coenzyme Q10, Coleus, Crataegus, Croton, Gynostemma, Radix polygalae, Tabebuia, Tribulus and Vitamin E together with pharmaceutically acceptable excipients.

2) The phytoceutical composition of claim 1, further comprising: Panax ginseng, Pfaffia paniculata, Rhapontium carthamoides, Rhodiola rosea, Andrographis paniculata, Astragalus membranaceus, Echinacea, Ganoderma lucidum, Grifola frondosa, Coenzyme Q10, Coleus forskohlii, Crataegus oxycantha, Croton lechleri, Gynostemma arial, Radix polygalae, Tabebuia avellanedae, Tribulus terrestris and Vitamin E together with pharmaceutically acceptable excipients.

3) The phytoceutical composition of claim 2, comprising the relative amounts of ingredients shown in Table 1, and optionally including water or gelatin.

4) A method of treating disease comprising administering an effective amount of the composition of claim 3 to a patient sufficient to alleviate said disease.

5) The method of claim 4, wherein the diseases are cardiac disorders and its symptoms, including—but not limited—to: Cardiac insufficiency, ischemic cardiopathy, obstructive hypertrophic myocardiopathy, congestive heart failure and hypertensive cardiopathy, high cholesterol and/or high triglyceride level.
